(19) United States
(12) Patent Application Publication
(10) Pub. No.: US 2019/0370932 A1
(43) Pub. Date: Dec. 5, 2019

(54) Systems And Methods For Transforming Media Artifacts Into Virtual, Augmented and Mixed Reality Experiences

(71) Applicant: Simon Romanus, Los Angeles (US)

(72) Inventor: Simon Romanus, Los Angeles (US)

(21) Appl. No.: 16/431,627

(22) Filed: Jun. 4, 2019

Related U.S. Application Data
(60) Provisional application No. 62/680,106, filed on Jun. 4, 2018.

(51) Int. Cl. G06T 3/00; G06T 15/20; H04N 13/111; G16H 30/40

(52) U.S. Cl. CPC G06T 3/0087; G06T 15/20; G06T 2210/41; G16H 30/40; H04N 13/111

(57) ABSTRACT

A virtual reality system comprising a media capturing device to capture one or more assets, the one or more assets corresponding to 2D and 3D ultrasound images and videos, and a processor communicatively coupled to the media capturing device to dynamically place the assets in a 3D virtual reality space.

### CROSS REFERENCE O RELATED APPLICATIONS

This application claims the benefit of U.S. Provisional Patent Application 62/680,106, filed Jun. 4, 2018.

### FIELD OF THE INVENTION

This invention relates generally to systems and methods for facilitating virtual and mixed reality and more specifically towards transforming media artifacts into virtual reality and mixed reality experiences.

### BACKGROUND OF THE INVENTION

Virtual reality (“VR”) is a three-dimensional computer-generated interface that allows users to see, move through and interact with information displayed as a three-dimensional world known as a virtual reality environment.

Augmented reality overlays digital information on real-world elements. Augmented reality keeps the real world central but enhances it with other digital details, layering new strata of perception, and supplementing your reality or environment.

Mixed reality brings together real world and digital elements. In mixed reality, you interact with and manipulate both physical and virtual items and environments, using next-generation sensing and imaging technologies.

Virtual, augmented or mixed reality environments can be created using libraries of media including images and video. Various systems and techniques exist for inserting media artifacts into a virtual, augmented or mixed reality environment. However, the design of these virtual, agumented or mixed reality environments presents numerous challenges, including the speed of the system in generating and delivering virtual content, quality of virtual content, and other system and optical challenges.

Thus, what is needed is a system to automate the process of capturing, building, rendering, delivering and distributing 2D images into the world of virtual and mixed reality.

### SUMMARY OF THE INVENTION

Embodiments of the present invention are directed to systems and methods for transforming media artifacts to virtual, augmented and mixed reality experiences for one or more users. In one embodiment, a system is provided that automates a delivery process of 2D and 3D images and video into a virtual reality and/or mixed reality space. The system also allows users to upload their digital pictures or videos on a website and have them automatically put into a virtual, augmented or mixed reality experience of their choice.

In one embodiment, a virtual reality system comprises a media capturing device to capture one or more assets, the one or more assets corresponding to 2D and 3D ultrasound images, and a processor communicatively coupled to the media capturing device to dynamically place the assets in a 3D virtual, augmented or mixed reality space. While this embodiment describes ultrasound images, it should be noted that the process may still be applied in other applications as well. That is, the service can allow for any image or video or object to be placed in the VR, AR or MR environment. The invention also gives the user the opportunity to choose background of his or her choice from a list of multiple images and videos.

One primary embodiment described within this application will be directed towards ultrasound images. In this embodiment, a virtual camera in the 3D virtual reality space is placed in the middle of a moving virtual “womb” model, wherein the womb model corresponds to the ultrasound images. In a further embodiment, there may also be other dynamic objects placed in the 3D virtual reality space such as, but not limited to, dust particles, animated rigged baby models and moving and animated dynamic lights.

Additional and other objects, features, and advantages of the invention are described in the detail description, figures and claims.

### BRIEF DESCRIPTION OF THE DRAWINGS

The following drawings illustrate an exemplary embodiment. They are helpful in illustrating objects, features and advantages of the present invention and the present invention will be more apparent from the following detailed description taken in conjunction with accompanying drawings in which:

FIG. 1 illustrates a system architecture of a virtual reality system interacting with one or more servers, according one illustrated embodiment.

FIG. 2 illustrates a process flow of a virtual reality system.

FIG. 3 is an image of an animated baby in womb via the virtual reality system.

FIG. 4 illustrates a user viewing a generated VR movie.

### DETAILED DESCRIPTION OF THE INVENTION

Reference will now be made in detail to the exemplary embodiments of the invention, examples of which are illustrated in the accompanying drawings. Whenever possible, the same reference numerals will be used throughout the drawings to refer to the same or like parts.

References to “one embodiment,” “at least one embodiment,” “an embodiment,” “one example,” “an example,” “for example,” and so on indicate that the embodiment(s) or example(s) may include a particular feature, structure, characteristic, property, element, or limitation but that not every embodiment or example necessarily includes that particular feature, structure, characteristic, property, element, or limitation. Further, repeated use of the phrase “in an embodiment” does not necessarily refer to the same embodiment.

Disclosed are methods and systems for transforming media artifacts to virtual reality (VR) augmented reality (AR) or mixed reality (MR) experiences. In one particular embodiment described herein, virtual content representing an ultrasound image may be strategically delivered to patients, medical professionals, and other users in a manner that is more immersive than the traditional way of looking at ultrasound images.

The following disclosure will provide various embodiments of such systems that may be integrated into a VR, AR or MR system. Although most of the disclosures herein will be discussed in the context of VR systems, it should be appreciated that the same technologies may be used for augmented and mixed reality systems as well. The following embodiments describe a novel process to facilitate the transformation of media artifacts into virtual, augmented and mixed reality experiences.

The present disclosure comprises systems and methods to automatically transform any 2D and 3D images and videos into a virtual reality, augmented reality or mixed reality experience. It can be appreciated that the methods and systems disclosed are automatic in their delivery process, delivering a rapid transformation from assets comprising ordinary 2D and 3D images and video to a virtual reality, augmented reality or mixed reality experience for the end user. According to an embodiment of the present disclosure, a user can upload digital pictures and videos onto a system website and have them automatically put into the virtual reality world of their choice and sent back to them as a complete 360/VR video clip.

In a preferred embodiment, a virtual reality system comprises a media capturing device to capture one or more assets, the one or more assets corresponding to 2D and 3D ultrasound images, and a processor communicatively coupled to the media capturing device to dynamically place the assets in a 3D virtual reality space. That is, the user upload digital assets corresponding to ultrasound images, and the system dynamically creates a 3D virtual reality space based off these virtual assets.

In a further embodiment, a virtual camera in the 3D virtual reality space is placed in the middle of a moving virtual “womb” model. In a further embodiment, there may also be other dynamic objects placed in the 3D virtual reality space such as dust particles, animated rigged baby models and moving and animated dynamic lights.

In an alternate embodiment, third party users can upload digital pictures and videos on a system website, wherein the system automatically places the digital media into a virtual reality world of the user's choice which is then delivered as a complete 360/VR video clip.

In one or more embodiments, the VR system comprises a computing network, comprised of one or more computer servers connected through one or more networking interfaces. The servers in the computing network may or may not be co-located. The one or more servers each comprise one or more processors for executing program instructions. The servers may also include memory for storing the program instructions and data that is used and/or generated by processes being carried out by the servers under direction of the program instructions. In one embodiment, the system server structure is hosted on AWS and uses a dynamic load balancer to handle different number of requests.

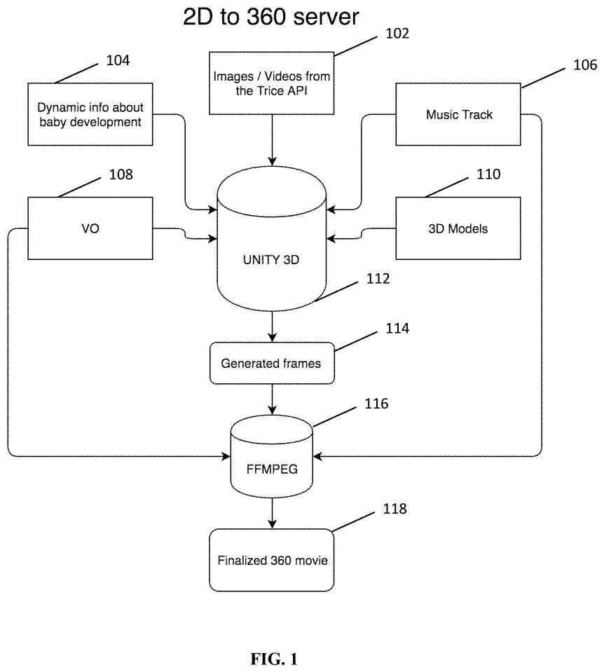

As disclosed in FIG. 1, data used to create a virtual reality experience may include, for example, dynamic info about baby development, ultrasound images/video, a music track, VO, 3D models, and other data used to define and/or describe a virtual environment. In one embodiment, data is imported from a cloud-based image collaboration service that enables clinics and hospitals to store, review and share medical images in a simple and cost efficient way.

In one embodiment, this data is fed into a UNITY 3D 112 environment, which then generates the frames 114. This is output into a FFMPEG file type 116, which is then used as a finalized 360 movie 118. In one embodiment, a prepared 3D scene made in Unity 3D imports the assets (also known as the images or film clips) and places them dynamically in the 3D space depending on the number of images or film clips. The system does this in a dynamic manor depending on the amount of assets and type of assets. Each film and image is presented as a texture on a plane in the 3D space and rendered using a proprietary shader that masks away the content near the edges of the material. Alternative embodiments may include video processing engines other than UNITY 3D.

Each frame is concatenated together into a movie using FFMPEG on the server. This happens after the main Unity Application has rendered all the required frames. A custom script instructs FFMPEG which files that needs to be concatenated and in what sequence. It can be appreciated that this is done on the system server side using pre-compiled codecs and renderers, thus speeding up the delivery of the 3D rendering.

According to an embodiment, the intro scene movie (which can be a 30-40-second-long animation of a fetus in a womb with a description of the current stage of the fetus) and the just created dynamic scene movie are concatenated using FFMPEG commands. This is made to save time and processing time on the server. Finally, the audio including the dynamic voice over and music track is muxed in (the audio files and video files are combined into one container file) together with the film clips and, according to a preferred embodiment, the final film is rendered out to the MP4 format.

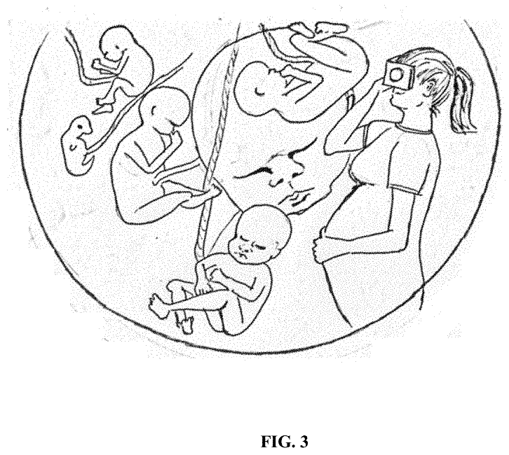

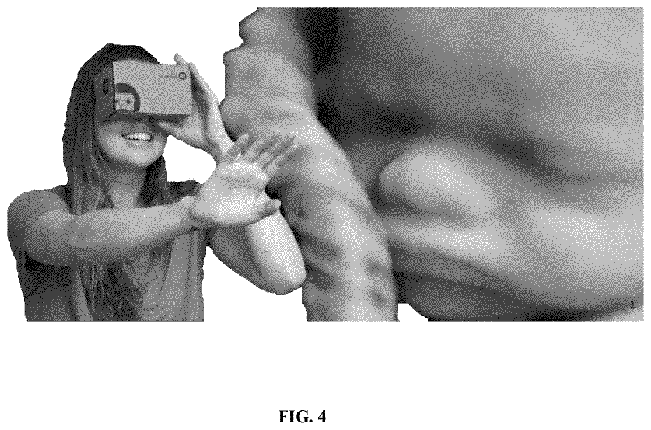

In one embodiment, the final film clip is saved into storage such as an Amazon S3 bucket. This storage can later serve the finalized files for whenever the user needs them, load balanced and distributed around the world. A URL for the film is saved in the local database together with the client ID. FIG. 3 shows an example of ultrasound images in a womb virtual reality space. The various media artificants are displayed in a VR type space. FIG. 4 shows a user viewing a finalized 360 womb movie via a VR headset.

In one embodiment, all temporary files used for building the final film on the server are deleted.

In yet another embodiment, the system sends out an email to the user with a custom URL to the page with the finalized 360 film. The finalized 360 movie can then be shown on a standard web browser on any device such as computer, mobile phone or tablet. It can also be downloaded and shared with any third party through a website or an app.

The final 360 movie clip can also be shown in both virtual reality with special VR goggles or as a 360 movie on a flat screen where a user can turn the picture around with a finger, mouse or by simply turning the phone or touchpad using its gyro.

In a further embodiment, a specific method for generating such a 360 movie is disclosed. The method comprises:
a) An end user is logged in to a website where they can see their ultrasound images and videos of their baby.
b) The end user is provided with an option of seeing these ultrasound images and videos in a virtual reality experience.
c) The end user can click on a link that will take them to a “Meet Your Baby” web site or app.
d) On the website or app the end user may receive relevant information about the service.
e) The end user is presented with an option to purchase the VR experience, and by clicking a link they will reach a paywall site.
f) On the paywall site, the user is presented with an information dialog where they can enter in their name, email address, week of pregnancy, gender and choice of music in the VR movie. In one embodiment, this information is stored in on a local database.
g) After filling in all necessary information they pay for the service.
h) The system grabs a selected number of maximum images and videos and puts them into a 360 spherical video. It also adds a dedicated animated video in the beginning showing information about the stage of the pregnancy that you are in. It adds the music of your choice and renders everything together to a VR/360 movie.
i) Once the rendering is complete and the movie is ready, the user will receive an email with a confirmation and a link to the movie.

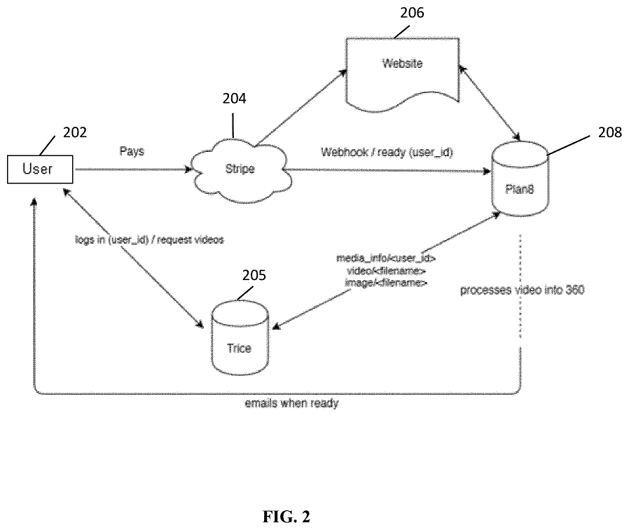

According to an embodiment, FIG. 2 discloses a workflow corresponding to a similar method. In one embodiment, when the user 202 has paid via stripe 204 for a service or plan 208 listed on a website 208 the system pulls in the images and/or film clips from the ultrasound, the files are stored temporarily on a local server. This is done using the provided Trice 205 id of the customer and then requesting the associated endpoint from the system servers. In alternative embodiments, other image sharing services may be used rather than Trice.

In one embodiment, a virtual camera in the 3D scene is placed in the middle of a moving virtual “womb” model. In the scene there may also be placed other dynamic objects such as dust particles, animated rigged baby models and moving and animated dynamic lights. In one embodiment, the scene is built using custom C# scripting, unique materials and rendering options.

In yet a further embodiment, the 3D scene is animated and a capture script captures each frame from the virtual 360 camera in the 3d scene and saves the frame image to a temporary folder on the server. The system utilizes a novel process that steps through each frame while updating all the components in the scene (such as movement, fades, particles and movement), all while the standard camera takes 6 screen shots in each direction of the “cube” surrounding the virtual camera. The system stitches together the sides of the cube into a “cubemap” image. The cubemap image can also later be converted into a equirectangular image.

Although the invention has been explained in relation to a preferred embodiment, it is to be understood that many other possible modifications and variations can be made without departing from the spirit and scope of the invention.

Various example embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Example aspects of the invention, together with details regarding technical components and architecture have been set forth above. As for other details of the present invention, these may be appreciated in connection with the above-referenced patents and publications as well as generally known or appreciated by those with skill in the art. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention

1. A method for generating a 360 movie, comprising:
capturing, by a media capturing device, one or more media assets from an end user, wherein the one or more media assets comprise 2D and 3D ultrasound images and video;
feeding the one or more media assets into a UNITY 3D environment;
generating, via the UNITY 3D environment, a plurality of frames;
outputting the plurality of frames into a FFMPEG supported file type, and then using the FFPEG supported file type as a finalized 360 movie.

2. The method of claim 1, further comprising:
authenticating the end user into a website, wherein the end user can see their ultrasound images and videos of their baby;
providing the end user an option of seeing these ultrasound images and videos in a virtual reality experience; pulling a selected number of maximum images and videos and putting it into an initial 360 spherical video;
adding the music of choice and rendering the music of choice together with the initial 360 spherical video to form a finalized movie.

3. The method of claim 1, wherein a virtual camera in the finalized 360 space is placed in the middle of a moving virtual “womb” model.

4. The method of claim 3, wherein the womb model corresponds to the 2D and 3D ultrasound images and video.

5. The method of claim 4, further comprising a capture script capturing each frame from the virtual 360 camera in a 3D scene and saving the frame image to a temporary folder on the server.

6. The method of claim 5, wherein the capture script further comprising stepping through each frame while updating all the components in the scene while the standard camera takes 6 screen shots in each direction of the “cube” surrounding the virtual camera.

7. The method of claim 6, further comprising stitching together the sides of the cube into a “cubemap” image.

8. A system, comprising:
a device connected to at least one processor; and
a non-transitory physical medium for storing program code and accessible by the device, wherein the program code when executed by the processor causes the processor to:
capture one or more media assets from an end user, wherein the one or more media assets comprise 2D and 3D images and video;
feed the media assets into a UNITY 3D environment;
generate, via the UNITY 3D environment, a plurality of frames;
output the plurality of frames into a FFMPEG supported file type, and then use the FFPEG supported file type as a finalized 360 movie.

9. The system of claim 8, wherein the program code when executed by the processor further causes the processor to: send the user an email with a confirmation and a link to the movie once the rendered movie is ready.

10. The system of claim 9, wherein the program code when executed by the processor further causes the processor to: place a virtual camera in the initial 360 spherical video in the middle of a moving virtual “womb” model.

11. The system of claim 9, wherein the program code when executed by the processor further causes the processor to: capture, via a capture script, each frame from the virtual 360 camera in a 3D scene and saving the frame image to a temporary folder on the server.

12. The system of claim 10, wherein the capture script further comprising stepping through each frame while updating all the components in the scene while the standard camera takes 6 screen shots in each direction of the “cube” surrounding the virtual camera.

13. The system of claim 11, wherein the program code when executed by the processor further causes the processor to stitch together the sides of the cube into a “cubemap” image.

14. A non-transitory computer-readable storage medium for dynamically placing media assets in a 3D virtual reality space, the storage medium comprising program code stored thereon, that when executed by a processor causes the processor to: capture, by a media capturing device, one or more media assets from an end user, wherein the one or more media assets comprise 2D and 3D images and video;
feed the media assets into a UNITY 3D environment;
generate, via the UNITY 3D environment, a plurality of frames;
output the plurality of frames into a FFMPEG supported file type, and then using the FFPEG supported file type as a finalized 360 movie.

15. The non-transitory computer-readable storage medium of claim 14, wherein the program code when executed by the processor further causes the processor to:
authenticate the end user into a website, wherein the end user can see their ultrasound images and videos of their baby;
provide the end user an option of seeing these ultrasound images and videos in a virtual reality experience;
pull a selected number of maximum images and videos and putting it into an initial 360 spherical video;
add the music of choice and rendering the music of choice together with the initial 360 spherical video to form a finalized movie.

16. The non-transitory computer-readable storage medium of claim 14, wherein the program code when executed by the processor further causes the processor to send the user will receive an email with a confirmation and a link to the movie once the rendered movie is ready.

17. The non-transitory computer-readable storage medium of claim 14, wherein the program code when executed by the processor further causes the processor to: place a virtual camera in the initial 360 spherical video in the middle of a moving virtual “womb” model.

18. The non-transitory computer-readable storage medium of claim 15, wherein the program code when executed by the processor further causes the processor to: capture, via a capture script, each frame from the virtual 360 camera in the 3d scene and saving the frame image to a temporary folder on the server.

19. The non-transitory computer-readable storage medium of claim 16, wherein the capture script further comprising stepping through each frame while updating all the components in the scene while the standard camera takes 6 screen shots in each direction of the “cube” surrounding the virtual camera.
